(19) United States
(12) Patent Application Publication
(10) Pub. No.: US 2008/0286286 A1
(43) Pub. Date: Nov. 20, 2008

(54) Methods to Treat Disease States by Influencing the Signaling of Ox-40-Receptors and High Throughput Screening Methods for Identifying Substances Therefor

(75) Inventors: Yong-Jun Liu, Pearland, TX (US); Tomoki Ito, Aichi (JP)

Correspondence Address: BRACEWELL & GIULIANI LLP, P.O. BOX 61389, HOUSTON, TX, 77208-1389, US

(73) Assignee: BOARD OF REGENTS, THE UNIVERSITY OF TEXAS SYSTEM, Austin, TX (US)

(21) Appl. No.: 11/659,266

(22) PCT Filed: Jan. 16, 2007
(86) PCT No.: PCT/US07/01228

§ 371 Date: Jun. 4, 2008

Related U.S. Application Data
(60) Provisional application No. 60/759,217, filed on Jan. 13, 2006.

(51) Int. Cl. A61K 39/395; G01N 33/535; A61P 37/02

(52) U.S. Cl. 424/172.1; 435/7.92

(57) ABSTRACT

OX40L inhibits the generation of IL-10-producing Tr1 cells from naïve and memory CD4+ T cells induced by the immunuosuppressive drugs dexamethasone and vitamin D3. This unique function of OX40L is not shared by two other costimulatory TNF-family members, GITR-ligand and 4-1BB-ligand. OX40L also strongly inhibits the generation of IL-10-producing Tr1 cells induced by two physiological stimuli provided by inducible costimulatory ligand and immature DCs and inhibits the production of IL-10 by regulatory T cells. It has thus been shown that signaling the OX40-receptor on human T cells by monoclonal antibodies, small molecules, or OX40L regulates the generation and function of IL-10 producing immunosuppressive T cells. Also provided are high throughput methods for identifying substances that promote or inhibit the generation and function of IL-10 producing T cells. Numerous disease states, such as human allergic, autoimmune, and autoimmune diseases, and cancer, may be treated by targeting OX40/OX40L.

### CROSS-REFERENCE TO RELATED APPLICATION

This non-provisional patent application claims the benefit under § 119(e) of U.S. Provisional Patent Application Ser. No. 60/759,217, filed Jan. 13, 2006, which is incorporated herein by reference.

### BACKGROUND OF THE INVENTION

1. Field of the Invention

This invention relates generally to immunology and, more specifically, to the treatment of disease states by influencing the signaling of OX40-receptors and high throughput screening methods for identifying substances therefor.

2. Description of Related Art

It has been proposed that interleukin (IL)-10-producing CD4⁺ type 1 regulatory T (Tr1) cells play a critical role in peripheral tolerance, in particular in limiting tissue damage to the host during inflammatory immune responses against different classes of microbial pathogens, including intracellular pathogens that preferentially induces TH1 immune responses or some extra-cellular parasites that preferentially induce TH2 immune responses. Indeed, the generation of IL-10-producing Tr1 cells appear to accompany both TH1 and TH2 immune responses in vivo and in vitro as demonstrated in previous and current studies.

Tr1 cells were originally isolated from patients with severe combined immunodeficiency who had undergone successful HLA-mismatched bone-marrow transplantation. Subsequently, IL-10-producing Tr1 cells were generated from naïve CD4⁺ T cells during antigen-driven T cell immune responses. IL-10-producing Tr1 cells are anergic in response to signaling through TCR, CD28 and IL-2 receptors and have the ability to suppress antigen-driven proliferation of naïve CD4⁺ T cells in vivo and in vitro. IL-10-producing Tr1 cells have the ability to inhibit the development of autoimmune diseases and limit the magnitude of immune responses to microbial pathogens.

The molecular signals that lead to the generation of IL-10-producing Tr1 cells have been extensively studied during the past decade. IL-10 and IFN-α have been described to induce the generation of IL-10-producing Tr1 cells from naïve CD4⁺ T cells activated through TCR and CD28. Most strikingly, a strategy to inhibit TH1 or TH2 differentiation by using anti-IL12 and anti-IL-4, together with a combination of dexamethasone (Dex) and the active form of vitamin D3 (vit D3), has been shown to induce the generation of large numbers of IL-10-producing Tr1 cells from naïve CD4⁺ T cells activated through TCR and CD28. In addition, immature dendritic cells (DCs) and DCs treated with IL-10 or IFN-α were shown to induce naïve CD4⁺ T cells to differentiate into IL-10-producing Tr1 cells. Signaling through the inducible costimulator (ICOS) on CD4⁺T cells by ICOS-ligand (ICOSL) also promoted their differentiation into IL-10-producing Tr1 cells.

However, little is known about the molecular signals that negatively regulate the generation of IL-10-producing Tr1 cells. Although immunosuppressive drugs, cytokines, costimulatory molecules, and DCs are implicated in the induction of Tr1 cells, signals that negatively regulate the generation of Tr1 cells remain elusive.

OX40/OX40-ligand (OX40L) represents a pair of costimulatory molecules critical for T cell proliferation, survival, cytokine production, and memory cell generation. Early in vitro experiments demonstrated that signaling through OX40 on CD4⁺ T cells lead to TH2, but not TH1 development. These results were supported by in vivo studies showing that blocking OX40/OX40L interaction prevented the induction and maintenance of TH2-mediated allergic immune responses. However, many studies reveal that blocking OX40/OX40L interaction ameliorated or prevented TH1-mediated diseases. Furthermore, administration of soluble OX40L or gene transfer of OX40L into tumors were shown to strongly enhance anti-tumor immunity in mice. Recent studies also suggest that OX40/OX40L may play a role in promoting CD8 T cell-mediated immune responses. A recent study also showed that OX40 signaling blocks the inhibitory function of CD4⁺CD25⁺ naturally occurring regulatory T cells. These together suggest that OX40/OX40L may play a critical role in the global regulation of peripheral immunity versus tolerance.

### SUMMARY OF THE INVENTION

It has been discovered that OX40L inhibits the generation and function of IL-10-producing Tr1 cells from naïve and memory CD4+ T cells induced by the immunosuppressive drugs dexamethasone and vitamin D3. It also has been discovered that OX40L inhibits the generation and function of IL-10 producing regulatory T cells. These discoveries demonstrate that signaling OX40 by OX40L suppresses the generation of human IL-10 producing immunosuppressive T cells in culture. This unique function of OX40L is not shared by two other costimulatory TNF-family members, GITR-ligand and 4-1BB-ligand. OX40L also strongly inhibits the generation and function of IL-10-producing Tr1 cells induced by two physiological stimuli provided by inducible costimulatory ligand and immature DCs. As one of skill in the art will recognize, it has thus been shown that signaling the OX40 receptor on human T cells by, for example, monoclonal antibodies, small molecules, or OX40L regulates the generation and function of IL-10 producing immunosuppressive T cells.

The discovery lends itself to numerous applications. For example, agonistic antibodies, small molecules, or OX40L could be used to suppress the generation and the function of IL-10 producing immunosuppressive T cells and therefore could be used to enhance immune responses to treat cancer and infectious diseases, or as an adjuvant for cancer vaccines. Antagonistic antibodies to OX40 or to OX40L, or antagonistic small molecules, could be used to enhance the generation and the function of IL-10-producing immunosuppressive T cells and therefore could be used for the development of therapies for autoimmune diseases and graft versus host diseases. The discovery also provides for high throughput methods for screening antibodies or small molecules either signaling OX40 or blocking OX40 signaling on T cells for the development of therapeutics for cancer, autoimmune diseases, and graft versus host diseases.

### BRIEF DESCRIPTION OF THE DRAWINGS

Some of the features and benefits of the present invention having been stated, others will become apparent as the description proceeds when taken in conjunction with the accompanying drawings, in which:

FIG. 1A is a plurality of graphs of the intracellular analysis of cytokine production by naïve CD4⁺ T cells by flow cytometry.

FIG. 1B is a plurality of graphs of cytokine production by naïve CD4⁺ T cells by ELISA.

FIG. 1C is a graph of suppressive function in T cells by [³H]thymidine incorporation.

FIG. 2A is a plurality of graphs of the intracellular analysis of cytokine production by memory CD4⁺ T cells by flow cytometry.

FIG. 2B is a graph of IL-10 production by memory CD4⁺ T cells by ELISA.

FIG. 3A is a plurality of graphs of the intracellular analysis of cytokine production by naïve CD4⁺ T cells by flow cytometry.

FIG. 3B is a graph of IL-10 production by naïve CD4⁺ T cells by ELISA.

FIG. 3C is a graph of the number of viable T cells counted.

FIG. 4A is a plurality of graphs of the intracellular analysis of cytokine production by naïve CD4⁺ T cells by flow cytometry.

FIG. 4B is a graph of IL-10 production by naïve CD4⁺ T cells by ELISA.

FIG. 4C is a plurality of graphs of the intracellular analysis of cytokine production by memory CD4⁺ T cells by flow cytometry.

FIG. 4D is a graph of IL-10 production by memory CD4⁺ T cells by ELISA.

FIG. 4E is a plurality of graphs of the intracellular analysis of cytokine production by naïve CD4⁺ T cells by flow cytometry.

FIG. 4F is a plurality of graphs of IL-10 production by naïve CD4⁺ T cells by ELISA.

FIG. 5 is a plurality of graphs of IL-10 production by regulatory T cells by ELISA.

### DETAILED DESCRIPTION

The present invention now will be described more fully hereinafter with reference to the accompanying drawings in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout.

It was discovered that a function of OX40L is the negative regulation of the generation of IL-10-producing Tr1 cells induced by immunosuppressive agents Dex and Vit D, ICOSL, or immature DCs. As one of skill in the art will recognize, this discovery demonstrates a general mechanism by which OX40L enhances immunity and breaks immunological tolerance.

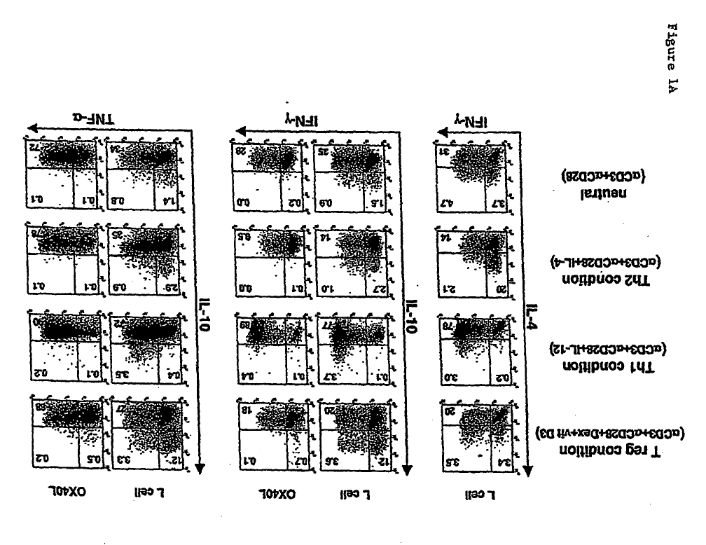

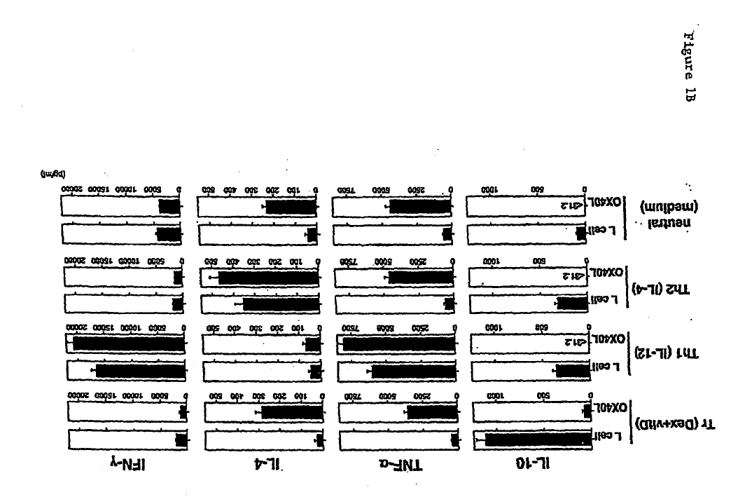

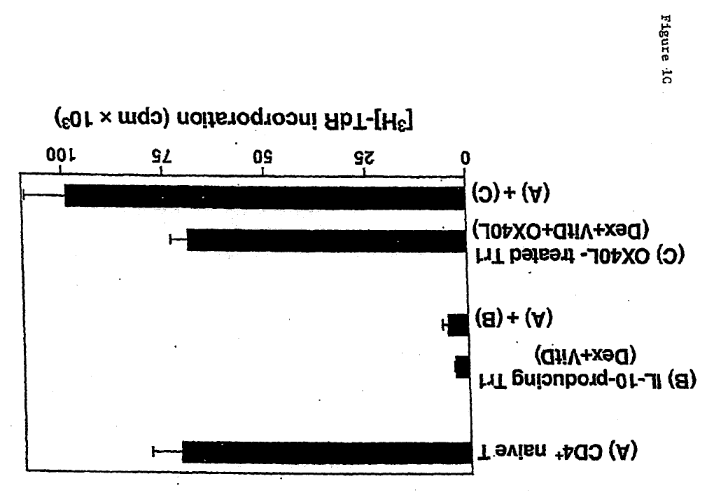

It was discovered that OX40L inhibits the generation of IL-10-producing Tr1 cells from CD4⁺ T cells induced by Dexamethasone and vitamin D3. It is known that a combination of the immunosuppressive drugs Dex and Vit D3 consistently induce the differentiation of naïve CD4⁺ T cells into IL-10-producing Tr1 cells. To investigate whether OX40L can inhibit the generation and function of IL-10-producing Tr1 cells, naïve CD4⁺ T cells were cultured with anti-CD3 plus anti-CD28 mAbs in the presence or absence of OX40L-transfected L cells in four different culture conditions including: 1) Tr1 (Dex and vit D3); 2) TH1 (IL-12); 3) TH2 (IL-4); or 4) neutral (medium alone) for 7 days (FIG. 1A). IL-10 production by the primed T cells was analyzed by intracellular cytokine staining and ELISA.

In the experiments of FIG. 1A, an intracellular analysis of cytokine production by naïve CD4⁺T cells was conducted by flow cytometry. Naïve CD4⁺T cells were cultured with anti-CD3 and anti-CD28 mAbs in the presence of IL-2 on parental L cells or OX40L-L cells with the indicated recombinant cytokines or reagents for 7 days. Percentages of the respective cytokine-producing T cells are indicated in each dot blot profile. The results show that OX40L inhibits the generation of IL-10-producing Tr1 cells from naïve CD4⁺ T cells induced by the different polarizing signals. As shown in FIG. 1A, between 2% to 4% of IL-10-producing Tr1 cells were generated from naïve CD4⁺T cells cultured in neutral or TH1 or TH2 conditions. More than 15% of IL-10-producing Tr1 cells were generated in culture with Dex plus vit D3. The addition of OX40L completely blocked the generation of IL-10-producing Tr 1 cells, while promoting the generation of TNF-α-producing T cells in all culture conditions.

These data were confirmed by ELISA data (FIG. 1B). In the experiments of FIG. 1B, cytokine production by naïve CD4⁺ cells was measured in supernatants after restimulation with anti-CD3 and anti CD28 mAbs for 24 h by ELISA. Native CD4⁺T cells were cultured with anti-CD3 and anti-CD28 mAbs in the presence of IL-2 on parental L cells or OX40L-L cells with the indicated recombinant cytokines or reagents for 7 days. The data are shown as mean ±SEM of four independent experiments. The results show that OX40L inhibits the generation of IL-10-producing Tr1 cells from naïve CD4⁺T cells induced by the different polarizing signals.

Naïve CD4⁺ T cells primed with Tr1 condition (Dex plus vit D3) were anergic and had the ability to suppress the proliferation of naïve CD4⁺T cells in response to anti-CD3 plus anti-CD28 mAbs (FIG. 1C). In the experiments of FIG. 1C, suppressive function in T cells was measured by [³H]thymidine incorporation. Mixtures of the indicated T cell populations were restimulated by anti-CD3 and anti-CD28 mabs. Error bars represent SEM of triplicate wells. It was discovered that naïve CD4⁺T cells primed with the same Tr1 condition in the presence of OX40L proliferated vigorously and failed to inhibit the proliferation of naïve CD4⁺T cells in response to anti-CD3 plus anti-CD28 mAbs. As understood by those of skill in the art, these data suggest that OX40L blocks the generation of functional Tr1 cells from naïve CD4⁺T cells induced by Dex and Vit D3.

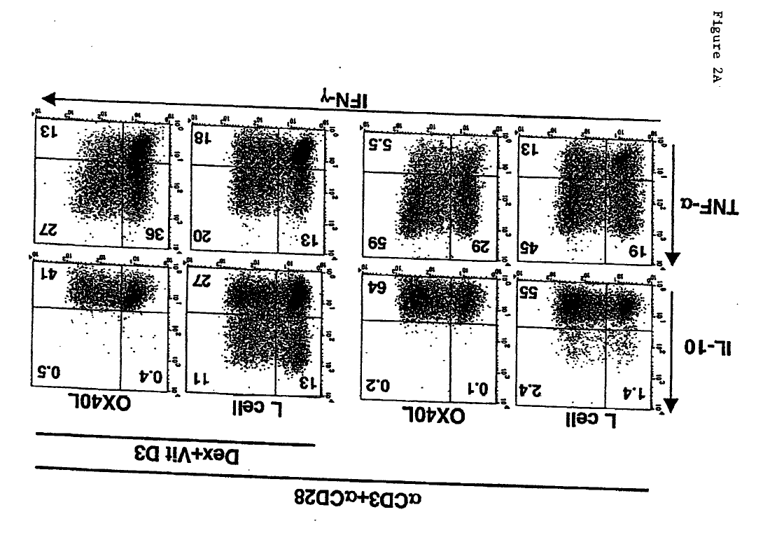

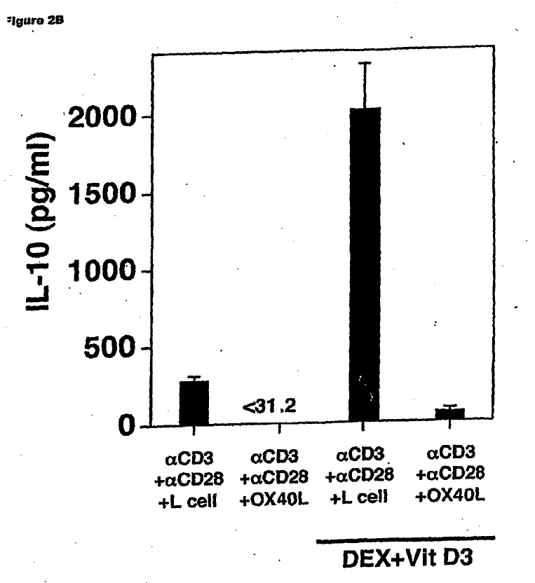

Also investigated was whether IL-10-producing Tr1 cells can be generated from memory CD4⁺CD45RA⁻CD45RO⁺ T cells, and whether OX40L can inhibit the generation of IL-10-producing Tr1 cells from memory CD4⁺T cells. Memory CD4⁺CD45RA⁻CD45RO⁺ T cells were cultured for 7 days with anti-CD3 plus anti-CD28 mAbs in the presence or absence of OX40L-transfected L cells Tr1 condition (Dex plus vit D3). In the experiments of FIG. 2A, an intracellular analysis of cytokine production by CD4⁺ memory T cells was conducted by flow cytometry. Memory CD4⁺CD45RO⁺CD25⁻ memory T cells were cultured with anti-CD3, anti-CD28 mAbs, and IL-2 on parental L cells or OX40L-L cells in the presence or absence of Dex plus vit D3 for 7 days. Percentages of the respective cytokine-producing T cells are indicated in each dot blot profile. The results show that OX40L inhibits the generation of IL-10-producing Tr1 cells from memory CD4⁺T cells under a condition with Dex plus Vit D3. FIG. 2A shows that large numbers of IL-10-producing cells (>20%) were generated from CD4⁺ memory T cells in culture with Dex plus vit D3. The addition of OX40L completely blocked the generation of IL-10-producing Tr1 cells and promoted generation of TNF-α-producing cells from memory CD⁴⁺ T cells.

The ability of Dex plus vit D3 to promote IL-10 production from memory CD4⁺T cells, and that this ability can be inhibited by OX40L, were confirmed by IL-10 ELISA analyses (FIG. 2B). In the experiments of FIG. 2B, IL-10 production by memory CD4⁺T cells was measured in supernatants after restimulation with anti-CD3 and anti-CD28 mAbs for 24 h by ELISA. The data are shown as mean ±SEM of four independent experiments. The results show that OX40L inhibits the generation of IL-10-producing Tr1 cells from memory CD4⁺ T cells under a condition with Dex plus Vit D3.

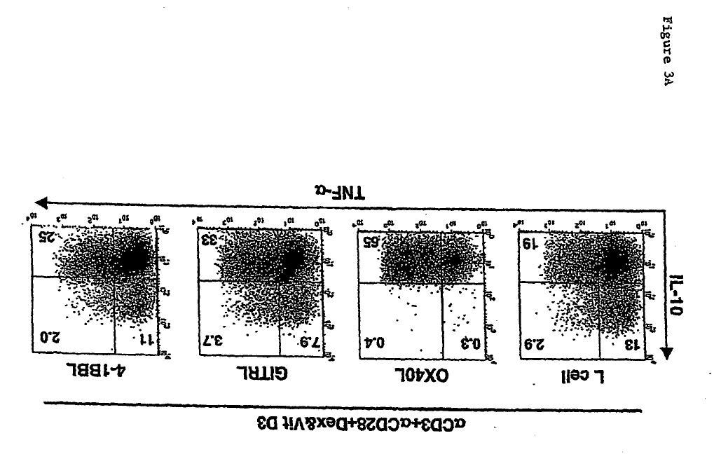

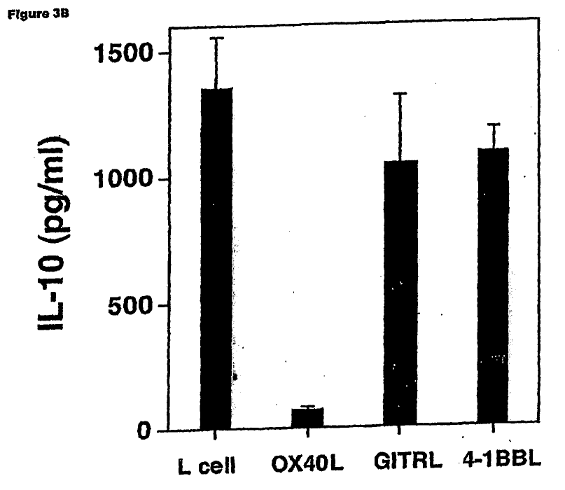

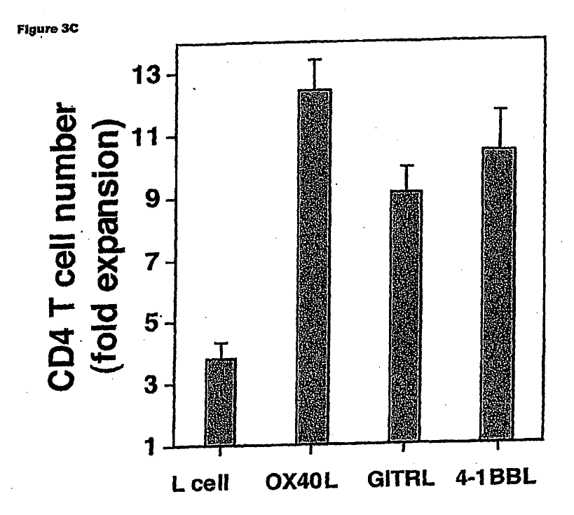

It was further discovered that OX40L inhibits the generation of IL-10-producing Tr1 cells, while other TNF-family members (GITRL and 4-1BBL) do not. Within the TNF-superfamily, OX40L, glucocorticoid-induced TNF receptor-ligand (GITRL), and 4-1BB-ligand (4-1BBL) have costimulatory-function for T cells. To investigate whether OX40L was unique in the inhibition of IL-10-producing Tr1 cells, naïve CD4⁺T cells were cultured with anti-CD3 plus anti-CD28 mAbs with Dex plus vit D3, with parental L′ cells or L cells transfected with OX40L, GITRL, or 4-1BBL for 7 days. While OX40L, GITRL, and 4-1BBL all promoted the generation of TNF-α-producing cells, only OX40L inhibited the generation of IL-10-producing Tr 1 cells (FIGS. 3A and B).

In the experiments of FIG. 3A, an intracellular analysis of cytokine production by naïve CD4⁺T cells was conducted by flow cytometry. Naïve CD4⁺T cells were cultured with anti-CD3, anti-CD28 mAbs, and IL-2 on parental L cells, OX40L-L cells, GITRL-L cells, or 4-1BBL-L cells in the presence of Dex plus vit D3 for 7 days. Percentages of the respective cytokine-producing T cells are indicated in each dot blot profile. The results show that OX40L but not GITRL nor 4-1BBL inhibits the generation of IL-10-producing Tr1 cells.

In the experiments of FIG. 3B, IL-10 by naïve CD4⁺ cells was measured in supernatants after restimulation with anti-CD3 and anti CD28 mAbs for 24 h by ELISA. The data are shown as mean ±SEM of four independent experiments. The results show that OX40L but not GITRL nor 4-1BBL inhibits the generation of IL-10-producing Tr1 cells.

OX40L, GITRL, and 4-1BBL all promoted the expansion of total T cell numbers (FIG. 3C). In the experiments of FIG. 3C, the number of viable T cells was counted. The data are shown as mean ±SEM of four independent experiments.

As understood by those of skill in the art, the results of FIG. 3A-C show that OX40L, but not GITRL nor 4-1BBL, inhibits the generation of IL-10-producing Tr1 cells. These data suggest that among the three members of TNF-superfamily known to costimulate T cells, OX40L has a novel and unique function in inhibiting the generation of IL-10-producing Tr1 cells.

It was further discovered that OX40L inhibits the generation of IL-10-producing Tr1 cells induced by ICOSL or immature DCs. ICOS and CD28 represent the two positive costimulatory receptors within the CD28 family expressed on T cells. Signaling through ICOS by agonistic Abs or ICOSL has been shown to promote CD4⁺T cells to produce IL-10. To investigate whether OX40L can inhibit the ability of ICOS to induce IL-10 production by CD4⁺T cells, naïve and memory CD4⁺T cells were cultured with anti-CD3 in the presence of ICOSL-transfected L cells, or ICOSL-transfected L cells in the presence of OX40L for 7 days.

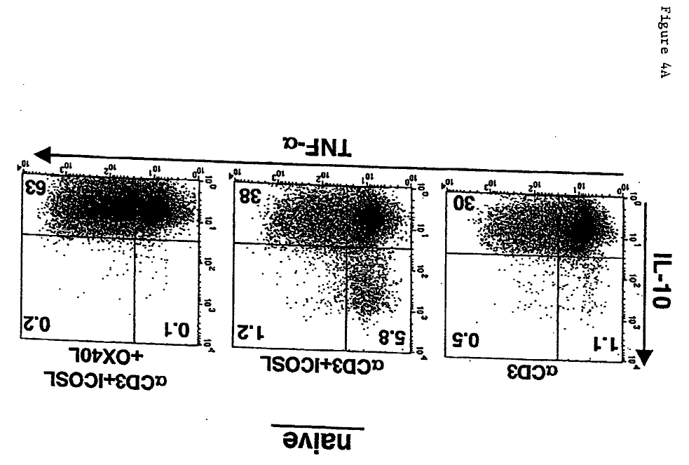

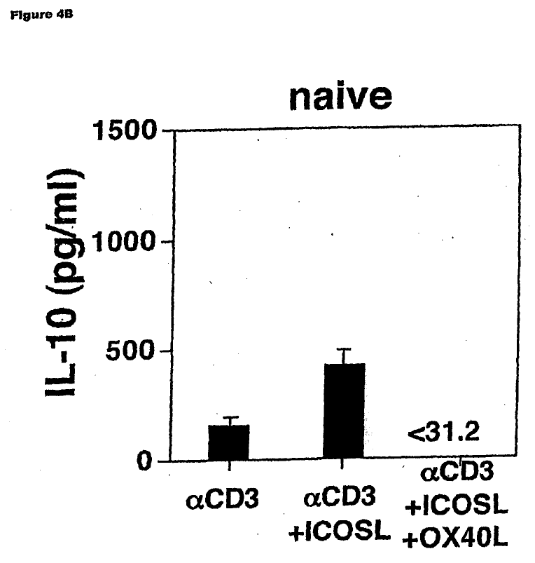

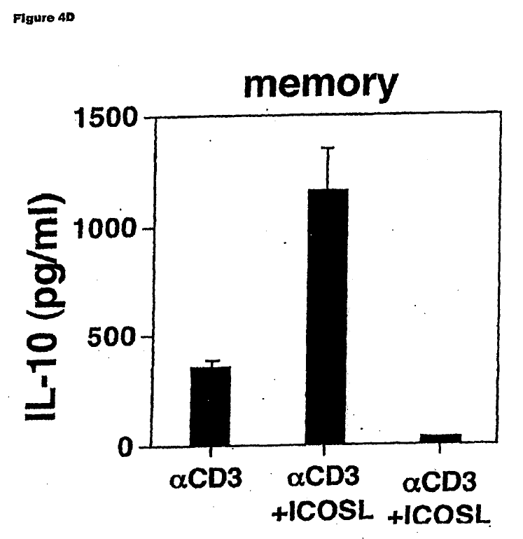

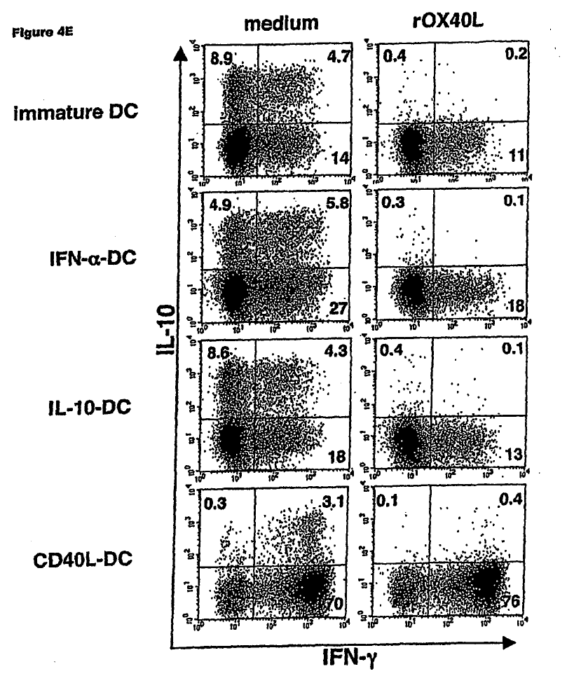

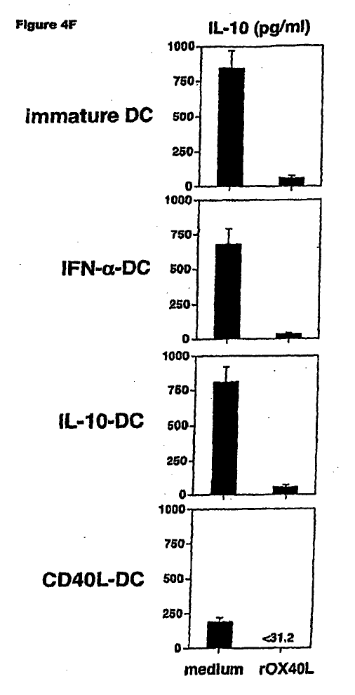

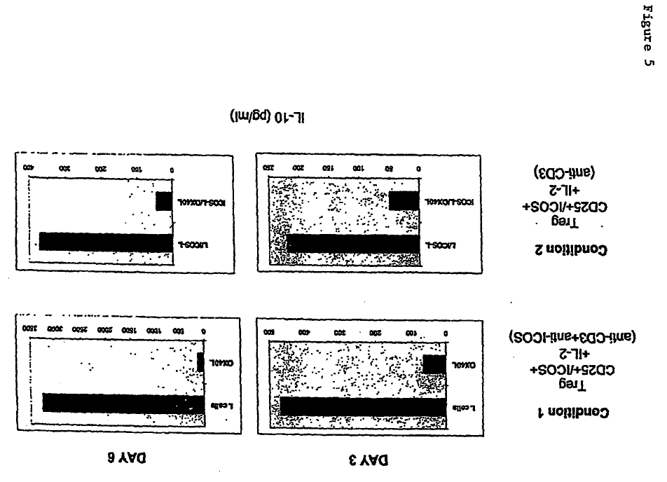

In the experiments of FIG. 4A, an intracellular analysis of cytokine production by naïve CD4⁺T cells was conducted by flow cytometry. Naïve CD4⁺T cells were cultured for 7 days on parental L cells, on a mixture of ICOSL-L cells and L cells, or on a mixture of ICOSL-L cells and OX40L-L cells, which were pre-coated with anti-CD3 mAb. Percentages of the respective cytokine-producing T cells are indicated in each dot blot profile. The results show that OX40L inhibits the generation of IL-10-producing Tr1 cells from naïve CD4⁺ T cells induced by ICOSL.

In the experiments of FIG. 4B, IL-10 production by naïve CD4⁺ cells was measured in supernatants after restimulation with anti-CD3 and anti-D28 mAbs for 24 h by ELISA. Naïve CD4⁺T cells were cultured for 7 days on parental L cells, on a mixture of ICOSL-L cells and L cells, or on a mixture of ICOSL-L cells and OX40L-L cells, which were pre-coated with anti-CD3 mAb. The data are shown as mean ±SEM of three independent experiments. The results show that OX40L inhibits the generation of IL-10-producing Tr1 cells from naïve CD4⁺T cells induced by ICOSL.

In the experiments of FIG. 4C, an intracellular analysis of cytokine production by memory CD4⁺T cells was conducted by flow cytometry. Memory CD4⁺T cells were cultured for 7 days on parental L cells, on a mixture of ICOSL-L cells and L cells, or on a mixture of ICOSL-L cells and OX40L-L cells, which were pre-coated with anti-CD3 mAb. Percentages of the respective cytokine-producing T cells are indicated in each dot blot profile. The results show that OX40L inhibits the generation of IL-10-producing Tr1 cells from memory CD4⁺ T cells induced by ICOSL.

In the experiments of FIG. 4D, IL-10 production by memory CD4⁺T cells was measured in supernatants after restimulation with anti-CD3 and anti-CD28 mAbs for 24 h by ELISA. Memory CD4⁺T cells were cultured for 7 days on parental L cells, on a mixture of ICOSL-L cells and L cells, or on a mixture of ICOSL-L cells and OX40L-L cells, which were pre-coated with anti-CD3 mAb. The data are shown as mean ±SEM of three independent experiments. The results show that OX40L inhibits the generation of IL-10-producing Tr1 cells from memory CD4⁺T cells induced by ICOSL.

The results of the experiments of FIG. 4A-D show that ICOSL significantly promoted the generation of IL-10-producing cells from both naïve and memory CD4⁺T cells. The addition of OX40L completely inhibited the generation of IL-10-producing cells from both naïve and memory CD4⁺T cells, while strongly promoting the generation of cells producing TNF-α.

It is known that immature DCs or DCs treated with IFN-α or IL-10 can induce naïve CD4⁺T cells to differentiate into IL-10-producing Tr1 cells. It was investigated whether OX40L could inhibit the generation of IL-10-producing Tr1 cells induced by DCs. As shown in FIG. 4E, immature DCs or DCs treated with IL-10 or IFN-α all induced the generation of more than 10% of IL-10-producing Tr1 cells from naïve CD4⁺T cells. By contrast, DCs activated by CD40L induce a strong TH1 response, accompanied by the generation of about 3% IL-10-producing Tr1 cells. Addition of recombinant OX40L in DC-T cell cultures completely inhibited the generation of IL-10-producing Tr1 cells induced by immature DCs and DCs treated with IL-10 and IFN-α. In addition, OX40L also inhibited the generation of the residual number of IL-10-producing Tr1 cells induced by the CD40L activated mature DCs. In the experiments of FIG. 4E, an intracellular analysis of cytokine production by CD4⁺ naïve T cells was conducted by flow cytometry. Naïve CD4⁺T cells were cocultured in the presence or absence of soluble recombinant OX40L for 7 days with immature DCs or DCs cultured with IFN-α, IL-10, and CD40L. Percentages of the respective cytokine-producing T cells are indicated in each dot blot profile. The results show that OX40L inhibits the generation of IL-10-producing Tr1 cells from CD4⁺T cells induced by DCs

The ability of OX40L to inhibit the generation of IL-10-producing Tr1 cells induced by DCs was confirmed by ELISA data FIG. 4F). In the experiments of FIG. 4F, IL-10 production by naïve CD4⁺ cells was measured in supernatants after restimulation with anti-CD3 and anti-CD28 mAbs for 24 h by ELISA. Naïve CD4⁺T cells were cocultured in the presence or absence of soluble recombinant OX40L for 7 days with immature DCs or DCs cultured with IFN-α, IL-10, and CD40L. The data are shown as mean ±SEM of three independent experiments. The results show that OX40L inhibits the generation of IL-10-producing Tr1 cells from CD4⁺T cells induced by DCs. Thus, these data demonstrate that OX40L could inhibit the generation of IL-10-producing Tr1 cells induced by more physiological signals provided by ICOSL and DCs.

It has been previously suggested that regulatory T cells are highly represented in the area of B cell non-Hodgkin's lymphoma and that B cells are involved in the recruitment of regulatory T cells into the area of the lymphoma. It was investigated whether influencing the signaling of OX40-receptors, such as by OX40L, could provide a therapy against B cell lymphoma. Frozen samples from B cell lymphoma patients were used to estimate the ability of OX40L to shut down IL-10 producing regulatory T cells. The samples used were follicular lymphoma obtained from a spleen specimen prior to any treatment. The cells were thawed, with 400×10⁶ frozen cells yielding 127×10⁶ live cells and 33.9×10⁶ dead cells (79% viability). A sufficient number of CD25+ cells were identified by FACS staining. In the experiments of FIG. 5, IL10 production by regulatory T cells was determined by ELISA. Regulatory T cells (Treg cells) were cultured under two different conditions. In condition 1, CD25+/ICOS+ cells were cultured with antiCD3 in the presence of IL-2 (900 μl/ml) on parental L cells or OX40L-L cells with anti-ICOS antibody for 3-6 days. In condiction 2, CD25+/ICOS+ cells were cultured with anti-CD3 in the presence of IL-2 (900 μl/ml) on ICOS-L-L cells or a mixture of OX40L-L can ICOS-L-L cells' for 3 to 6 days. Cytokine production was measured in the supernatants by ELISA. The results show that OX40L greatly inhibited IL-10 production by Treg cells.

The present findings, that OX40L has the capacity to inhibit the generation and function of IL-10-producing Tr1 cells induced by the immunosuppressive drugs Dex plus vit D3, ICOSL, or DCs, highlights a novel mechanism by which OX40L promotes immunity and breaks tolerance during different forms of CD4- or CD8-mediated immune responses, as would be understood by one of skill in the art. The ability of OX40L to inhibit the generation of IL-10-producing Tr1 cells during both IL-12 induced TH1 or IL-4 induced TH2 responses suggest that OX40L may control the magnitude of TH1- or TH2-mediated immune responses. Furthermore, the ability of OX40L to inhibit the generation of IL-10-producing Tr1 cells appears to be a unique property of OX40L, because the two other TNF-family members GITRL and 4-1BBL do not have this functional property. Moreover, the ability of OX40L to inhibit IL-10 production by Treg cells identifies OX40L as a potent treatment for B cell lymphoma and other cancers.

Many molecules have been identified that promote the generation of IL-10-producing Tr1 cells, including IL-10, IFN-α, ICOSL, and immunosuppressive compounds such as Dex plus vit D3. OX40L represents a potent inhibitor for the generation of IL-10-producing Tr1 cells not only from naïve CD4⁺T cells, but also from memory CD4⁺T cells and regulatory T cells. This novel property of OX40/OX40L may explain a recent report showing that OX40 signaling allows anergic autoreactive T cells to acquire effector cell functions. Targeting OX40/OX40L thus provides for treatments for human allergic and autoimmune diseases and as well as for the development of treatments for human infectious diseases and cancer.

The present discoveries also provide for high throughput screening methods. More specifically, and as understood by those skilled in the art, high throughput methods to screen for antagonistic or agonistic monoclonal antibodies or small molecules that bind to OX40-receptors, and that can inhibit the generation and function of IL-10 producing cells or promote the generation and function of IL-10 producing cells, are made possible. In one such method, a human T cell line (SU-DHL-1) having the ability to produce IL-10 was transfected with the human OX40-gene (SUOX40). 100,000 SUOX40 cells were cultured with either 100,000 mouse fibroblast cells (L cells) or 100,000 mouse fibroblast cells expressing the human OX40-ligand (OX40-ligand L cells) in 96 well-plates. After 48 hours of culture, culture supernatants were collected for the measurement of IL10 by IL-10-specific ELISA. In a representative experiment, 100,000 SUOX40 cells produced up to 6,000 pg/ml IL-10 cultured in the absence of OX40-ligand. In the presence of OX40-ligand, 100,000 SUOX40 cells produced less than 1,000 pg/ml IL-10. This culture method may be used to screen for, inter alia, antagonistic monoclonal antibodies or small molecules that block the ability of OX40-ligand to inhibit IL-10 production by SUOX40 cells. Alternatively, this culture method may be modified by replacing OX40-ligand expressing L cells with potential agonistic monoclonal antibodies or small molecules specific to OX40 to determine, inter alia, their ability to inhibit IL-10 production by SUOX40 cells.

The following materials and methods were used:

L cell lines. Human GITRL, OX40L, 4-1BBL, ICOSL expressing L cells were generated by retroviral mediated transduction, as understood by those of skill in the art. Briefly, full-length coding sequence for human GITRL (Accession# NM_{—}005092), OX40L (Accession# NM_{—}003326), 4-1BBL (Accession# NM_{—}003811), ICOSL (Accession# NM_{—}015259) was amplified by RT-PCR with RNA prepared from HSV-1 stimulated PBMCs. Subsequently the cDNAs were cloned into an MSCV based retroviral vector pMIGW2 and the resulting plasmids were verified by restriction enzyme digestion and DNA sequencing. To produce recombinant retroviruse, each vector was co-transfected with packaging constructs pCL-gp (gag/pol) and pHCMV-VSVg (VSV glycoprotein envelop) in HEK293T cells. Two days later, the virus containing culture supernatants were harvested and used to infect CD32 L cells at moi 100. Under this condition >95% cells were productively transduced.

Generation of monocyte-derived DCs. Isolated CD14⁺ monocytes (purity >94%) were cultured in the presence of 100 ng/ml GM-CSF and 50 ng/ml IL-4 (both from R&D) for 5 days, as understood by those of skill in the art. The resulting immature DCs were washed and cultured for 24 h with IFN-α (1000 U/ml, PBL Biomedical Laboratories), IL-10 (10 ng/ml, R&D), and irradiated CD40L-transfected L cells (DC to L cell ratio, 4:1) to obtain mature DCs, as understood by those of skill in the art.

CD4⁺T cell stimulation. Naïve CD4⁺T cells and memory CD4⁺T cells (each purity >99%) were isolated from PBMCs using CD4⁺T cell Isolation Kit II (Miltenyi Biotec) followed by cell sorting (CD4⁺CD4⁺RA⁺CD45RO⁻CD25⁻ fraction as naïve T cells and CD4⁺CD45RA⁻CD45RO⁺CD25⁻ fraction as memory T cells), as understood by those of skill in the art 4×10⁴ freshly purified allogeneic naïve CD4⁺T cells were cocultured with immature or cultured DCs (DC to T ratio, 1:10) in the presence or absence of recombinant human OX40L (R&D, 100 ng/ml) in round-bottomed 96-well culture plates for 7 days, as understood by those of skill in the art. Purified CD4⁺T cells were also cultured with IL-12 (10 ng/ml, R&D), IL-4 (25 ng/ml, R&D), or combination of dexamethasone (5×10⁻⁸ M, Life Technologies) and 1alpha,25-dihydroxyvitamin D3 (10⁻⁷ M) for 7 days in the presence of soluble anti-CD28 mAb (CD28.2, 1 μg/ml) and IL-2 (50 U/ml, R&D) on the irradiated CD32/OX40L-L cells, CD32/GITRL-L cells, CD32/4-1BBL-L cells, or parental CD32-L cells which had been pre-coated with anti-CD3 mAb (OKT3, 0.2 μg/ml) in 48-well culture plates (T cell to L cell ratio, 2.5:1), as understood by those of skill in the art. In some experiments, CD4⁺T cells were cultured for 7 days on the CD32-L cells, mixture of CD32-L cells and CD32/ICOSL-L cells (ratio 1:1), or mixture of CD32/ICOSL-L cells and CD32/OX40L-L cells (ratio 1:1) pre-coated with anti-CD3 mAb (0.2 μg/ml) in 48-well culture plates, as understood by those of skill in the art. RPMI 1640 was used and supplemented with 10% FCS, 2 mM L-glutamine, 1 mM sodium pyruvate, penicillin G, and streptomycin for the cultures, as understood by those of skill in the art.

Analyses of T cell cytokine production. The cultured T cells were collected and washed, and then restimulated with plate-bound anti-CD3 (5 μg/ml) and soluble anti-CD28 (2 μg/ml) at a concentration of 1×10⁶ cells/ml for 24 h, as understood by those of skill in the art. The levels of IL-4, IL-10, TNF-α, and IFN-γ in the supernatants were measured by ELISA (all kits from R&D), as understood by those of skill in the art. For intracellular cytokine production, the cultured T cells were restimulated with 50 ng/ml of PMA plus 2 μg/ml of ionomycin for 6 h. Brefeldin A (10 μg/ml) was added during the last 2 h, as understood by those of skill in the art. The cells were stained with a combination of PE-labeled mAbs to IL-4 or TNF-α, FITC-labeled mAbs to IFN-γ, and APC-labeled anti-IL-10 (all from BD) using FIX and PERM kit (CALTAG), as understood by those of skill in the art.

T cell expansion and suppressive function assay. T cells were collected and resuspended in an EDTA-containing medium to dissociate the clusters, as understood by those of skill in the art. Viable cells were counted by trypan-blue exclusion of the dead cells, as understood by those of skill in the art. For suppressive function assay, naïve CD4⁺T cells (A) and Tr1 cells generated from naïve CD4⁺T cells by anti-CD3 mAb, anti-CD28 mAb, IL-2, Dex, and vit D3 in the presence of parental L cells (3) or OX40L-L cells (C), these three cell types and their mixtures at a 1:1 ratio were then restimulated for 5 days by culturing in the presence of 5 μg/ml anti-CD3 mAb and 1 μg/ml anti-CD28 mAb, after which time the cellular proliferation was assessed by [³H]thymidine incorporation, as understood by those of skill in the art.

In the drawings and specification, there have been disclosed embodiments of the invention and, although specific terms are employed, they are used in a generic and descriptive sense only and not for the purpose of limitation, the scope of the invention being set forth in the following claims.

1. A method to treat a disease state comprising the step of administering to a person in need thereof a therapeutically effective amount of a substance that influences IL-10 production by T cells.

2. The method of claim 1 wherein the substance is an antibody agonistic to an OX40-receptor.

3. The method of claim 1 wherein the substance is a small molecule agonistic to an OX40-receptor.

4. The method of claim 1 wherein the substance is OX40L.

5. The method of claim 1 wherein the substance is an antibody antagonistic to an OX40-receptor or OX40L.

6. The method of claim 1 wherein the substance is a small molecule antagonistic to an OX40-receptor or OX40L.

7. The method of claim 1 wherein the disease state is selected from the group consisting of autoimmune diseases, graft versus host diseases, cancer, and infectious diseases.

8. The method of claim 1 wherein the disease state is B cell lymphoma.

9. The method of claim 1 wherein the T cell is a regulatory T cell.

10. The method of claim 1 wherein the T cell is a memory T cell.

11. The method of claim 1 wherein the substance influences IL-10 production by T cells by inhibiting the production and function of IL-10 producing immunosuppressive T cells.

12. A method to treat B cell lymphoma comprising the step of administering to a person in need thereof a therapeutically effective amount of a substance that inhibits the production of IL-10 by regulatory T cells.

13. The method of claim 12 wherein the substance is an antibody agonistic to an OX40-receptor.

14. The method of claim 12 wherein the substance is a small molecule agonistic to an OX40-receptor.

15. The method of claim 12 wherein the substance is OX40L.

16. A high throughput method to screen for substances that influence the generation or function of IL-10 producing cells comprising the steps of:
(A) transfecting T cells having the ability to produce IL-10 with an OX40-gene;
(B) culturing the transfected T cells with fibroblast cells and a substance of interest;
(C) collecting the culture supernatants; and
(D) analyzing the IL10 content of the culture supernatants.

17. The method of claim 16 wherein the method is used to screen for antagonistic monoclonal antibodies or small molecules that block the ability of an OX40-ligand to inhibit IL-10 production, wherein the fibroblast cells express the OX40-ligand, and wherein the substance of interest is a potential antagonistic monoclonal antibody or small molecule.

18. The method of claim 16 wherein the method is used to screen for agonistic monoclonal antibodies or small molecules specific to an OX40-receptor that inhibit IL-10 production and wherein the substance of interest is a potential agonistic monoclonal antibody or small molecule.

19. The method of claim 16 wherein the T cells are human T cells and the fibroblast cells are mouse fibroblast cells.

20. The method of claim 16 wherein the IL-10 content of the culture supernatants is analyzed using IL-10 specific ELISA.
